(19) United States
(12) Patent Application Publication
(10) Pub. No.: US 2006/0120915 A1
(43) Pub. Date: Jun. 8, 2006

(54) Sterilization apparatus, and method for sterilizing surfaces

(76) Inventor: John E. Lewandowski, Cypress, TX (US)

Correspondence Address: Peter L. Brewer, Patent Attorney, 4609 Crossover Lane, Memphis, TN, 38117, US

(21) Appl. No.: 11/007,554

(22) Filed: Dec. 8, 2004

(51) Int. Cl. A61L 2/10

(52) U.S. Cl. 422/24; 422/3

(57) ABSTRACT

A sterilization apparatus is provided. The sterilization apparatus generally includes an ultraviolet light source, a socket for receiving the ultraviolet light source, and a light sensor. The light sensor is configured to actuate the ultraviolet light source where there is little or no light in an area. The light sensor further deactivates the ultraviolet light source upon sensing light. A method of sterilizing a surface in an area using ultraviolet radiation is also provided. In one embodiment, the method comprises the steps of detecting the degree of light intensity within an area; if light is below a designated level of intensity, causing an ultraviolet light source to be activated; detecting the degree of light intensity within the area a second time; and, if light is above a designated level of intensity, causing the ultraviolet light source to be deactivated. Preferably, the ultraviolet light source is ultraviolet-C radiation.

### BACKGROUND OF THE INVENTION

1. Field of the Invention

The present invention relates to an apparatus and method for sterilizing an enclosed area. The invention also relates to an apparatus and method for sterilizing one or more surfaces in an enclosed area.

2. Description of the Related Art

Ultraviolet (UV) light has been used for disinfection and sterilization of objects. An example is the use of UV light in barber shops for the sterilization of combs and cutting instruments. Ultraviolet light may be produced artificially by electric-arc lamps. Ultraviolet light may also be generated by mercury bulbs. Such light sources have been used for disinfection and sterilization of objects.

Ultraviolet light generated is short-wavelength radiation with a wavelength of 400 nanometers or less. One type of ultraviolet light is “UV-C” light. UV-C is a high frequency wavelength of light within the ultraviolet band. Certain UV-C light has a wavelength of between 100 and 280 nm and has been shown to have bactericidal effects. The availability of low to medium pressure mercury bulbs that generate UV-C light has led to the development of devices which use “UV-C” to decontaminate air and water supplies.

U.S. Pat. No. 6,656,424 discloses a method for sterilizing hospital or surgical areas using ultraviolet radiation. U.S. Patent Application No. 2002/0031460 provides an air cleaning and disinfecting system that employs an array of UV lamps. U.S. Patent Application No. 2003/0217641 teaches an air filtration apparatus that includes a UV light sterilization chamber for destroying airborne pathogenic bacteria. While these and other patent documents teach the use of ultraviolet radiation to sterilize air, it is desirable to be able to employ ultraviolet light to disinfect more specific areas and surfaces. Examples of such areas include residential bathrooms, public restroom facilities, and public lavatories. It is also desirable to be able to sterilize objects such as eating utensils, medical instrumentation and cooking equipment. At the same time, it is desirable to apply UV light in such a manner as to avoid exposure to humans or other mammals.

U.S. Patent Application No. 2004/0025899 shows a canister that receives toothbrushes. The canister includes an internal ultraviolet bulb for sanitizing toothbrushes once they are received into the canister. However, the device is limited to the sterilization of toothbrushes placed within the device.

Accordingly, a need exists for a sterilization apparatus for cleaning fixtures in residential bathrooms and in public restrooms. In addition, a need exists for an apparatus that safely cleans eating utensils, medical instrumentation, cooking equipment and other devices. Further, a need exists for a method of disinfecting surfaces in residential bathrooms and public restrooms.

### SUMMARY OF THE INVENTION

A sterilization apparatus is first provided. The sterilization apparatus generally includes an ultraviolet light source; a socket for receiving the ultraviolet light source; and a light sensor configured to (a) actuate the ultraviolet light source upon detection of an absence of light at a designated intensity for a specified period of time, and (b) deactivate the ultraviolet light source upon sensing light above the designated intensity level. The apparatus may be battery powered. Alternatively, the sterilization apparatus may further include electrical leads for placing the apparatus in electrical communication with an electrical outlet.

In one aspect, the sterilization apparatus includes a cover for directing the UV light source in a desired direction. Optionally, the apparatus further includes a pivoting connector between the ultraviolet light source and the socket which enables a user to more specifically direct the ultraviolet light source towards a surface. Preferably, the ultraviolet light source emits ultraviolet-C radiation.

A method of sterilizing an area using ultraviolet radiation is also provided. In one embodiment, the method comprises the steps of directing an ultraviolet light source at the surface of an object in the area; detecting the degree of light intensity within an area a first time; if light is below a designated level of intensity, causing an ultraviolet light source to be activated; detecting the degree of light intensity within the area a second time; and, if light is above the designated level of intensity, causing the ultraviolet light source to be deactivated. Preferably, the light sensor is part of a sterilization apparatus such as the apparatus described above. The apparatus again may be battery powered, or may include leads for placing the sterilization apparatus in electrical communication with an electrical outlet. Preferably, the ultraviolet light source is again ultraviolet-C radiation.

An alternate method of sterilizing an object in an area using ultraviolet radiation is also provided. The method includes the steps of (a) directing an ultraviolet light source at the object in the area; (b) detecting the degree of light intensity within the area a first time; (c) if light is above a designated level of intensity, causing the ultraviolet light source to be deactivated, while if light is below a designated level of intensity, causing an ultraviolet light source to be activated; (d) detecting the degree of light intensity within the area a second time; (e) if light is above a designated level of intensity, causing the ultraviolet light source to be deactivated, while if light is below a designated level of intensity, detecting the presence of motion in the area; and (f) if motion in the area is detected, causing the ultraviolet light source to be deactivated.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 provides a perspective view of an apparatus for use in sterilizing an enclosed area, or a surface within an enclosed area.

FIG. 2 is a flow chart showing steps for practicing one embodiment of the methods herein.

FIG. 3 is a flow chart showing steps for practicing sterilization methods in an alternate embodiment.

### DETAILED DESCRIPTION

Definitions

As used herein, the term “sterilization” or “sterilize” or “sterile” means rendering an organism incapable of reproduction.

The term “light source” means a radiation source that generates ultraviolet light.

The terms “ultraviolet light” and “ultraviolet radiation” are synonymous, and refer to radiation having a wavelength or wavelengths between 200 nm and 400 nm. One non-limiting example is UV-C light.

The term “light sensor” means any sensor capable of detecting a prevailing light intensity.

The terms “actuated” or “actuation” mean both to cause something to be actuated, or to allow something to remain actuated. Similarly, the term “deactivated” means to either cause something to be deactivated or to remain deactivated.

### DESCRIPTION OF SPECIFIC EMBODIMENTS

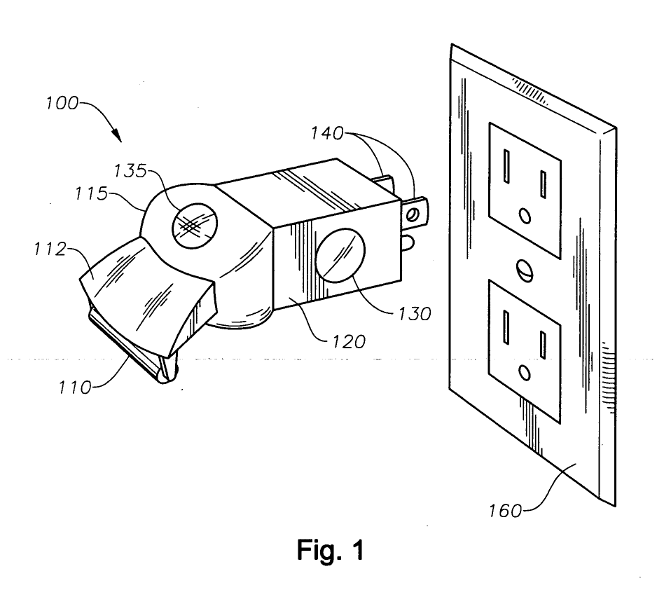

FIG. 1 provides a perspective view of a sterilization apparatus 100 for use in sterilizing a surface within an area. The surface (not shown) may be on a fixture or device in a bathroom such as a faucet, a handle, a sink bowl, a toilet bowl, a urinal, a light switch or other objects. The surface may alternatively be on a utensil used in rendering dental or medical treatment. The surface may alternately comprise an eating utensil or cooking hardware.

The sterilization apparatus first comprises an ultraviolet light source 110. The ultraviolet light source 110 emits ultraviolet radiation. Preferably, the ultraviolet light source 110 emits ultraviolet-C radiation, or “UV-C light.” A non-limiting example of a UV light source is a mercury bulb. UV bulbs are commonly available, and are manufactured by such companies as Sylvania and General Electric.

The sterilization apparatus 100 next includes a socket 120. The socket 120 is configured to receive the ultraviolet light source 110. The socket 120 also includes electrical circuitry for the apparatus 100. A cover 112 [John—I need to add 112 to FIG. 1] may extend from the socket 120 as an aid in directing UV light towards a desired surface. The cover 112 is preferably fabricated from polycarbonate plastic, light aluminum, or other material that is substantially non-translucent.

As a further aid in directing UV light, the cover 112 may wrap substantially around the light source 110, but then have one or more openings such as louvers or a window. Optionally, louvers or windows could be selectively opened or closed to direct UV light. Alternatively, the cover 112 may only block UV light across a radius of 120 to 180 degrees. In this arrangement, and as shown in FIG. 1, the sterilization apparatus 100 includes a pivoting connector 115. The connector 115 is placed between the ultraviolet light source 110 and the socket 120. The pivoting connector 115 permits the user to rotate the light source 110 and cover 112 to direct the ultraviolet light towards a specific surface. For example, the user might direct the light source more directly towards a toilet seat, a faucet handle, a light switch or other washroom item so as to sanitize that object and the air around it. The ultraviolet light will kill or otherwise neutralize pathogens in the air and on bathroom (or other) surfaces.

The sterilization apparatus 100 next includes a light sensor. The light sensor is shown schematically at 130 in FIG. 1. The light sensor 130 detects ambient light or light intensity within an area. The light sensor is part of an electrical circuitry (not shown) that permits electric power to activate the light source 110. If the detected degree of light intensity is below the designated level, the sterilization apparatus 100 actuates the ultraviolet light source 110. However, if the detected degree of light intensity is above the designated level, the sterilization apparatus 100 deactivates the ultraviolet light source 110. Where the ultraviolet light source 110 was already off, then the light sensor 130 will not actuate the light source 110, but the light source 110 will remain off.

One example of a light sensor that may be employed is a CdS (Cadmium Sulfide) photoresistor, or CdS photocell. In this photocell, the resistance of the sensor varies based on the amount of light that hits it. In one arrangement, the resistance can vary from 300K in the dark to 1 K resistance in the light. Thus, the more light that is present, the better the electricity is conducted through the circuit. In this way, the photocell's resistive value is used to provide electrical power to the light source 110 in response to the degree of light present. A transistor is preferably included in the circuit to amplify the current that flows through the CdS cell.

Other light-responsive devices may be employed, such as photosensors and light-to-voltage converters. In this respect, the present inventions are not limited by the type of light sensor employed, so long as the light sensor senses light in an area and is used to actuate the ultraviolet light source 110 upon detection of light below a designated level, and to deactivate the light source 110 upon detection of light above a designated level.

Actuation and deactivation of the light source 110 may also be accomplished through use of control circuitry that also includes relays, switches, clocks or other electronic devices. In this respect, and by way of example only, the light source may be connected to a timer. The timer would shut off the UV light source after a designated period of time, such as ten minutes. Thus, if the detected light intensity is low, then the light source 110 will be actuated for so long as the detected light intensity remains low or for ten minutes, whichever is shorter. In this arrangement, the timer may also prevent the light source 110 from actuating more than once in any given time frame, such as within four hours.

The timer is preferably part of a control circuit. The control circuit may be implemented on an integrated circuit, or with discrete components. Thus, the control circuit may shut off the light source 110 after a designated time period, such as ten minutes. The control circuit may also prohibit the light source 110 from being reactivated for a designated period of time, such as four hours.

The sterilization apparatus 100 is preferably battery powered. This permits the apparatus 100 to be placed in a cabinet or a drawer or on a shelf. So long as the cabinet doors or the drawer is shut, the level of light in the “area” will remain low enough to enable the UV light source to be actuated. In the residential context, kitchen cupboards and kitchen/bathroom drawers remain closed most of the time. When the door or drawer is opened and then re-closed, the light source 110 may turn on for a preset length of time optimum for killing germs and to sterilize and sanitize the contents of the cabinet or drawer. After the preset time expires, the sterilization apparatus 100 will shut off. If the drawer or door is opened before the preset time expires, then the outside light source (acting through the light sensor 130) will turn off the UV light 110. The photoelectric sensor 130 will reset the timer once again.

Where the sterilization apparatus 100 is battery powered, it may be configured to be secured to the wall of a cabinet or to the side of a drawer. In an alternate arrangement, the sterilization apparatus 100 may include leads for placing the apparatus 100 in direct electrical communication with an electrical outlet. In the arrangement of FIG. 1, leads are shown at 140. The leads 140 are exploded away from an outlet 160. The depicted outlet 160 is a standard three-prong outlet designed to facilitate 120-volt AC power with a connection to ground. However, the scope of the present inventions is not limited to the type of power or the configuration of the outlet, if any, used. The leads 140 in FIG. 1 are integral to the apparatus 100. However, it is understood that the leads 140 could be attached to a plug at the end of a power cord (not shown).

A method for sterilizing a surface using ultraviolet radiation is also provided. In one embodiment, the method generally includes the first step of detecting the degree of light intensity within an area a first time. The area may be either a public or private area. For example, the area may be a washroom where bodily functions occur or where personal hygiene is practiced. Alternatively, the area may be a drawer or cabinet in a home or in a doctor's or dentist's office. A light sensor is employed for this detection step. If the detected intensity of light in the area is below a designated level, the light sensor causes or otherwise permits electrical power to activate the ultraviolet light source 110.

Next, the degree of light intensity within the area is detected a second time. Again, the light sensor 130 is employed for this second detecting step. If the degree of light intensity remains below the designated level, then the light source 110 remains activated. If, on the other hand, the degree of light intensity detected is above the designated level of intensity, the light sensor 130 causes the ultraviolet light source 110 to be de-activated.

In performing the above methods, it is preferred that the light sensor 130 be part of a sterilization apparatus, such as the apparatus 100 described above. This would include the control circuit having the timer features. Where the apparatus 100 includes the pivoting connector 112, then an additional step of redirecting the ultraviolet light source 110 towards a surface within the area may be performed.

It is preferred that the second detecting step be repeated frequently. For example, the sensor 130 could be configured with the control circuit to detect light every 0.25 seconds. In this way, an individual who enters the area and turns on a light source will cause the light sensor 130 to be very quickly deactivated.

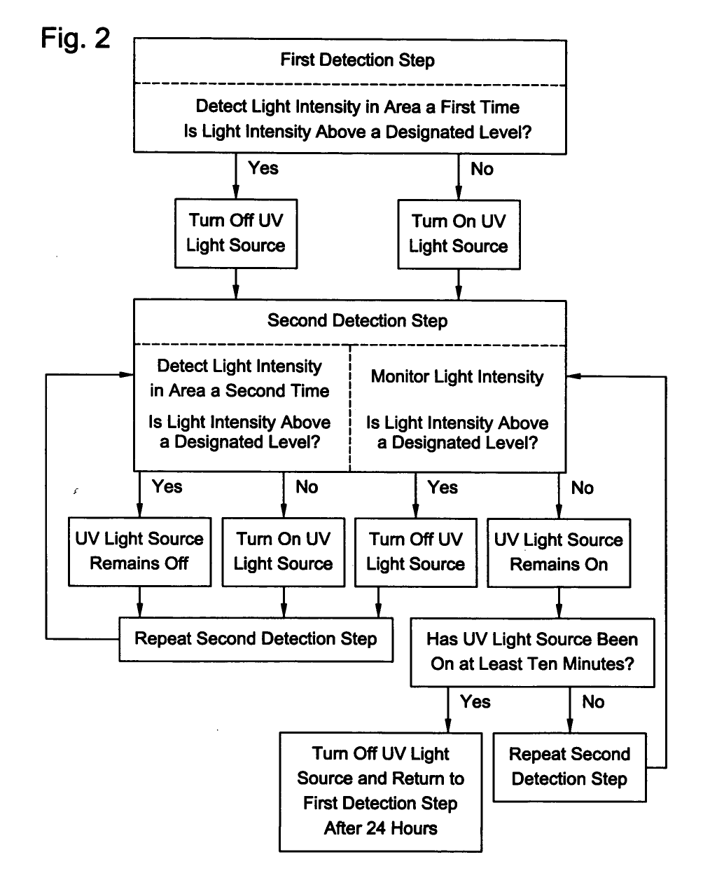

FIG. 2 provides a flow chart demonstrating the above sterilization steps and features, in one embodiment. In FIG. 2, first and second detection steps are shown. In this embodiment, when power is supplied to the sterilization apparatus 100, the light source 130 and the light sensor 130 are immediately actuated. In all likelihood, the user will be “plugging in” or otherwise powering the apparatus 100 in a lighted room. The light sensor 130 will immediately sense light above a designated level of light intensity, and cause the light source 110 to be immediately turned off. However, if light intensity below the designated level is prevailing, then the UV light source 110 will be turned on.

As noted above, the light sensing feature preferably senses light intensity in the area frequently, e.g., every 0.25 seconds. Therefore, the light sensor 130 will quickly perform the second detection step. If the light intensity in the area remains above the designated intensity level, then the UV light 110 will remain off. If, however, the light intensity in the area has dropped below the designated intensity level, then the UV light will be turned on. In either instance, the second detection step is repeated quickly.

In the instance where the detected light intensity in the area is below the designated level, then the light source 110 will remain actuated. A clock is preferably provided with the apparatus 100 to measure the length of time in which the light source 110 has been on. During subsequent detection steps, the apparatus will check the clock. When the UV light source 110 has been on for a designated length of time such as ten minutes, then the light source 110 will be directed by the control circuit to turn off. The clock will then be reset. The clock will prohibit the light sensor 130 from turning on the light source 110 for an extended period of time, such as 24 hours, regardless of the readings of the light sensor 130.

As yet an additional feature of the sterilization apparatus 100, a motion sensor 135 may be included. The motion sensor would be tied into the control circuit. Where the motion sensor detects motion in the area such as from a human or from a pet, then the motion sensor 135 will override the light sensor 130 and cause the light source 130 to be deactivated.

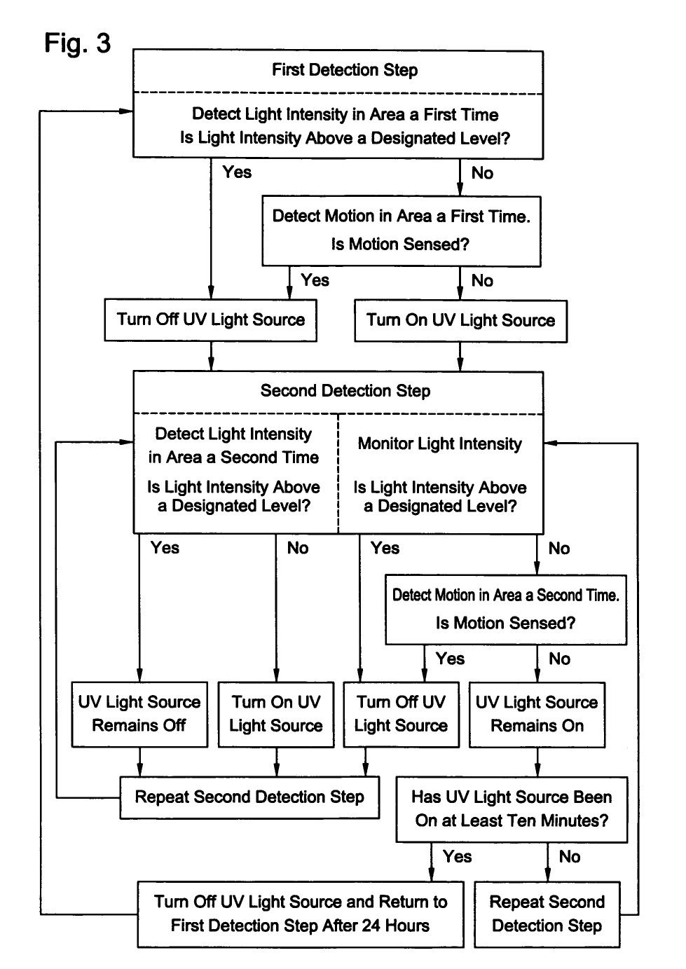

FIG. 3 provides a flow chart demonstrating the above sterilization steps and features, in an alternate embodiment. In FIG. 3, a first detection step and a second detection step are again shown. However, in this embodiment, the detection steps include not only the detection of light intensity, but the detection of motion as well. In this manner, a human or pet that enters a room that is lit below the designated level of light intensity will nevertheless cause the UV light source 110 to be turned off.

In the flow chart of FIG. 3, power is again supplied to the sterilization apparatus 100. At that point, the light source 110, the light sensor 130 and the motion sensor 135 are all immediately actuated. In all likelihood, the user will be “plugging in” or otherwise powering the apparatus 100 in a lighted room. The light sensor 130 will immediately sense light above a designated level of light intensity, and “turn off” the light source 110. If light intensity below the designated level is prevailing, the UV light source 110 will not remain on if the motion sensor 135 senses motion.

The second detection step quickly follows. As noted above, the light sensing feature preferably senses light intensity in the area frequently, e.g., every 0.25 seconds. Therefore, the light sensor 130 will quickly perform the second detection step. If the light intensity in the area remains above the designated intensity level, then the UV light 110 will remain off. If, however, the light intensity in the area has dropped below the designated intensity level, then the UV light will be turned on. However, this occurs only if no motion is sensed by the motion sensor 135. If motion is sensed, then the UV light is turned off and the second detection step is repeated.

In the instance where the detected light intensity in the area is below the designated level and no motion is sensed in the area, then the light source 110 will remain actuated. A clock is preferably provided with the apparatus 100 as part of a control circuit to measure the length of time in which the light source 110 has been on. During subsequent detection steps, the apparatus will check the clock. When the UV light source 110 has been on for a designated length of time such as ten minutes, then the light source 110 will be directed to turn off. The clock will then be reset. The clock will prohibit the light sensor 130 from turning on the light source 110 for an extended period of time, such as 24 hours, regardless of the readings of the light sensor 130 and the motion sensor 135.

I claim:

1. A sterilization apparatus for operation in an area, comprising:
an ultraviolet light source;
a socket for receiving the ultraviolet light source; and
a light sensor to sense light in the area and to actuate the ultraviolet light source upon detection of light below a designated level, and to deactivate the light source upon detection of light above a designated level.

2. The sterilization apparatus of claim 1, further comprising:
leads for placing the sterilization apparatus in electrical communication with an electrical outlet.

3. The sterilization apparatus of claim 1, wherein:
the sterilization apparatus is battery powered.

4. The sterilization apparatus of claim 1, further comprising:
a cover for at least partially covering the light source so as to direct ultraviolet radiation.

5. The sterilization apparatus of claim 4, further comprising:
a pivoting connector between the ultraviolet light source and the socket so as to direct the ultraviolet light source towards a surface.

6. The sterilization apparatus of claim 1, wherein:
the ultraviolet light source emits ultraviolet-C radiation.

7. The sterilization apparatus of claim 1, further comprising:
a control circuit having a timer that shuts off the ultraviolet light source after a designated period of time regardless of the level of light sensed by the light sensor.

8. The sterilization apparatus of claim 7, wherein:
the control circuit further prevents the ultraviolet light source from being actuated more than once during a designated period of time regardless of the level of light sensed by the light sensor.

9. The sterilization apparatus of claim 1, further comprising:
a motion sensor that causes the ultraviolet light source to be deactuated regardless of the level of light sensed by the light sensor.

10. A method of sterilizing a surface in an area using ultraviolet radiation, comprising the steps of:
(a) directing an ultraviolet light source at the surface of an object in the area;
(b) detecting the degree of light intensity within the area a first time;
(c) if light is below a designated level of intensity, causing an ultraviolet light source to be activated;
(d) detecting the degree of light intensity within the area a second time; and
(e) if light is above a designated level of intensity, causing the ultraviolet light source to be deactivated.

11. The method of sterilizing an area of claim 10, wherein the light sensor is part of a sterilization apparatus comprising:
an ultraviolet light source;
a socket for receiving the ultraviolet light source; and
a light sensor, the light sensor being configured to actuate the ultraviolet light source upon detection of light below a designated level, and to deactivate the light source upon detection of light above a designated level.

12. The method of claim 11, wherein the sterilization apparatus further comprises:
leads for placing the sterilization apparatus in electrical communication with an electrical outlet.

13. The method of claim 11, wherein:
the sterilization apparatus is battery powered.

14. The method of claim 11, wherein the sterilization apparatus further comprises:
a cover for at least partially covering the light source so as to direct ultraviolet radiation.

15. The method of sterilizing an area of claim 14, wherein:
the sterilization apparatus further comprises a pivoting connector between the ultraviolet light source and the socket so as to further direct the ultraviolet light source towards a surface; and
the method further comprises the step of rotating the pivoting connector so as to further direct the ultraviolet light source towards a surface within the area.

16. The method of sterilizing an area of claim 10, wherein the sterilization apparatus further comprises:
the ultraviolet light source emits ultraviolet-C radiation.

17. The method of claim 11, wherein the sterilization apparatus further comprises:
a control circuit having a timer that shuts off the ultraviolet light source after a designated period of time regardless of the level of light sensed by the light sensor.

18. The method of claim 17, wherein:
the control circuit further prevents the ultraviolet light source from being actuated more than once during a designated period of time regardless of the level of light sensed by the light sensor.

19. The method of claim 1, wherein the sterilization apparatus further comprises:
a motion sensor that causes the ultraviolet light source to be deactuated regardless of the level of light sensed by the light sensor.

20. A method of sterilizing an object in an area using ultraviolet radiation, comprising the steps of:
(a) directing an ultraviolet light source at the object in the area;
(b) detecting the degree of light intensity within the area a first time;
(c) if light is above a designated level of intensity, causing the ultraviolet light source to be deactivated, while if light is below a designated level of intensity, causing an ultraviolet light source to be activated;
(d) detecting the degree of light intensity within the area a second time;
(e) if light is above a designated level of intensity, causing the ultraviolet light source to be deactivated, while if light is below a designated level of intensity, detecting the presence of motion in the area; and
(f) if motion in the area is detected, causing the ultraviolet light source to be deactivated.
